(19) United States
(12) Patent Application Publication
(10) Pub. No.: US 2018/0021167 A1
(43) Pub. Date: Jan. 25, 2018

(54) COMPACT PERINEAL WARMING DEVICE FOR PERSONAL NON-INVASIVE PORTABLE AND STATIONARY USE TO PREVENT AND ALLEVIATE PROSTATE DISCOMFORT

(71) Applicant: RODOLFO FERNANDEZ, SR., KINGS MOUNTAIN, NC (US)

(72) Inventors: RODOLFO FERNANDEZ, SR., KINGS MOUNTAIN, NC (US); OSVALDO DIAZ, ORLANDO, FL (US); RODOLFO FERNANDEZ, JR., KINGS MOUNTAIN, NC (US)

(21) Appl. No.: 15/624,965

(22) Filed: Jun. 16, 2017

Related U.S. Application Data
(60) Provisional application No. 62/365,005, filed on Jul. 21, 2016.

(51) Int. Cl. A61F 7/02; A61F 7/00

(52) U.S. Cl. CPC A61F 7/02; A61F 7/007; A61F 2007/0048; A61F 2007/0071; A61F 2007/0228; A61F 2007/0282; A61F 2007/008; A61F 2007/0081; A61F 2007/0082; A61F 2007/0088

(57) ABSTRACT

Compact perineal warming device for personal non invasive portable and stationary use to prevent and alleviate prostate discomfort, to use without any kind of assistance at home, the workplace or while driving, wearing it concealed under or exposed over the clothes, comprising a small warming unit integrated by an encased ceramic electrical resistance, which is attached to a special strap-on supporting harness or to a special adjustable split cushion to keep it at the user's perineum in order to radiate concentrated infrared heat focused towards the prostate, being preset to locally increase the temperature within safe limits to prevent and relieve the pain and discomfort associated with some common disorders of the prostate which is easily operated and powered at safe low voltage from a portable power bank or from any available power source through conventional power adapters.

### REFERENCES CITED

U.S. PATENT DOCUMENTS

| | | | |
|---|---|---|---|
| 2,842,655 | July 1958 | SCHWEBEL | 219/527 |
| 3,016,446 | January 1962 | KALBACH | 219/528 |
| 3,407,818 | October 1968 | COSTANZO | 128/384 |
| 3,465,120 | September 1969 | MERNA | 219/211 |
| 4,201,218 | May 1980 | FELDMAN ET AL. | 607/112 |
| 4,279,255 | July 1981 | HOFFMAN | 607/112 |
| 4,932,950 | June 1990 | JOHNSON | 604/392 |
| 5,286,089 | February 1994 | GOLDMAN | 297/452 |
| 6,006,136 | December 1999 | GLUCKSMAN | 607/98 |
| 6,308,341 B1 | October 2001 | SHELTON | 2/400 |
| 6,648,909 B2 | November 2005 | HELMING | 607/108 |
| 6,868,290 | March 2005 | BOLMSJO | 607/101 |
| 7,014,652 B2 | March 2006 | CIOANTA ET AL. | 607/105 |
| 7,526,344 B2 | April 2009 | KIM | 607/138 |
| 9,039,084 | May 2015 | LUCAS | 297/257 |

### CROSS REFERENCE TO RELATED APPLICATIONS

This application claims the benefit of U.S. Provisional Application No. 62/365,005, filed on Jul. 21, 2016

### STATEMENT REGARDING FED. SPONSORED R & D

(Not Applicable)

### REFERENCE TO SEQUENCE LISTING

(Not Applicable)

### BACKGROUND OF THE INVENTION

### Field of the Invention

The present invention is related to therapeutic human body heating devices in general, and particularly to a device intended to increase within safe limits the temperature of the male prostate gland by means of a non invasive external electrical heating device for use directly by the patient himself without further assistance.

Hyperthermia medical treatments, which are based on the increasing of temperature of the human body as a whole, in part or of local specific parts, organs or tissues, has been utilized in therapeutic treatments of many different ailments since ancient times, using a great variety of heating procedures and heat sources that have been evolving according to the scientific and technical advances.

There are two general ways to apply the hyperthermia treatments: either using external heating sources such as hot air, hot water, water steam, fluid filled pads, electricity, radiation, infrared waves, radio waves, micro waves, magnetic induction, ultrasound, laser, etc., or internally inserting heating electrodes or probes directly into or near the tissues or organs to be treated.

In particular, internal and external hyperthermia treatments have been successfully used to prevent and alleviate or relieve prostate pain and other discomfort symptoms that frequently affect adult men, being usually caused by diseases such as Prostatitis and Benign Prostate Hyperplasia (BPH) as well as Prostate Cancer.

Among other home external treatments to alleviate the prostate diseases symptoms, hot water seat bath has been traditionally one of the most commonly used and recommended hyperthermia treatments.

Thus for long time, pain and discomfort relief treatments consisting in hot water seats and Jacuzzis have been very popular for the relatively common human male prostate disorders, but such treatments are bothersome, require relatively bulge, complex and costly equipments and installations, and cause relatively long waste of time and energy, heating large portion of the body in uncomfortable ways.

Therefore, due to the practical advantages of the use of human body local heating devices, some effective alternate home use devices as well as many medical internal and invasive therapeutic devices have been developed to relieve prostate disorders symptoms, relying on the use of hyperthermia therapeutic treatments by means of heat sources to locally increase the temperature of the prostate gland tissue.

Consequently, many patents have been issued for this kind of devices and a great variety of designs has been introduced in this field, regarding their components and operation, with diverse shapes and sizes, different features and degrees of complexity.

According to our search, most patents have been always related to relatively complex invasive devices that must be handled and operated by qualified specialists in medical facilities, requiring its insertion through the rectum or through the urethra, some even requiring highly qualified assistance, while others much less effective, are either relatively large or bulky electric heating pads, or chemical hot-cold pads requiring periodical maintenance, replacement and restoring processes.

### Prior Art Patents

Some examples of prior art patents that somehow may relate to the present invention are mentioned and briefly discussed below.

The U.S. Pat. No. 6,006,136 issued on December 1999 to Gluksman, discloses a Heating Pad consisting in an electric pliable heating pad adaptable to the surface of the human body and used to relieve local pain symptoms, but it spread the heat over relatively extended areas and is not very practical to use for the prostate.

The U.S. Pat. No. 6,308,341 B1 issued on October 2001 to Shelton, discloses a Temperature and Compression Treatment Underpants consisting in a gel-pack attached to an underpants and heated or cooled to place in the genital, perineal or anal areas with a strap to press the pad to the skin.

The U.S. Pat. No. 6,648,909 B2 issued on November 2003 to Helming, discloses a Solo Perineal Hot and Cold Pack consisting in a device for providing thermal therapy to the perineal and anal areas of a patient, mainly for alleviating women post-partum pain.

In those previous mentioned prior art inventions the temperature is not constant, but varies with time not providing a stable beneficial effect to the user, while there are some hygienic inconveniences, as well as some additional handling procedures for their use.

The U.S. Pat. No. 6,868,290 issued on March 2005 to Bolmsjo discloses a Thermotherapy catheter and method of prostate thermotherapy with improved guide and heat confinement which like most of the other inventions for thermotherapy of the prostate is an invasive sophisticated thermotherapy instrument for use in medical facilities and operated by qualified health practitioners.

The U.S. Pat. No. 7,014,652 B2 issued on March 2006 to Cioanta et al., discloses Methods for Treat Prostatitis which teaches a complex invasive medical device to be used by a physician or health qualified technician in a medical facility.

Said prior art invention includes the use of a balloon which is inserted into the bladder through the urethra, and then expanding and contracting with inflation media, at the same time applying heat to a portion of the prostatic urethra, so it is highly invasive and cannot be used at home by the patient himself.

The U.S. Pat. No. 4,201,218 issued to Feldman et al. in May 1980, discloses a Therapeutic Heat Application consisting in a portable heating device with electronic control and comprising a liquid filled component; thus, despite being portable, it is more complex and less suitable than the object of the present invention to be placed in the perineal area for heating the prostate gland, particularly when the user is in sitting position.

The U.S. Pat. No. 4,279,255 issued on July 1981 to Hoffman, discloses a Localized Body Heat Applicator Device which is also a portable device consisting in a heating flexible band to heat body parts, that is powered by a battery pack with electronic control.

The purpose of said prior invention is to heat the skin in localized areas, and according to what is claimed, its heating means comprises a flexible unit having a plurality of distinct sets of electric resistance elements, in order to accommodate to the irregular surface of the skin in different parts of the human body.

Said prior art has a more general use, is much more complex and less reliable than the object of the present invention, and the heating unit is flexible, a feature that while very useful in general for its adaptation to the shape of different parts of the body, is a disadvantage in particular to be used and pressed to the skin at the perineal area for external hyperthermia treatment of the prostate.

There are many other prior art inventions in this field like the examples mentioned above to apply hyperthermia medical treatment to the prostate, but mostly by means of relatively complex and invasive devices that must be inserted into the body and handled by medical specialists in medical facilities like hospitals, clinics and physicians' offices, and therefore are not portable whatsoever and cannot be used directly by the patient himself at home.

The U.S. Pat. No. 4,932,950 issued on Jun. 2, 1990 to Johnson, discloses a Self-adjusting suspension system for perineal shield, comprising a waistband portion with one or more elastic tensioning strips and two elastic strips that embrace around the user's thighs.

Said invention above, includes a common basic design typical of all strap-on systems, but substantially different to the strap-on supporting harness comprised within the scope of the present invention, since each invention related to perineal devices suspending systems features specific differentiated elements according to its intended particular function.

In the case of the suspension system within the present invention, the main differentiating element is the sort of sliding little fabric bridge with touch fastening means to attach onto it the warming unit.

The U.S. Pat. No. 9,039,084 issued on May 2015 to Lucas, discloses a Golf cart prostate seat, comprising a motorized movable seat cushion to be installed in golf carts to protect the prostate area of the driver.

While said adjustable cushion provides useful and convenient features for the prostate protection, it is relatively sophisticated and is particularly focused for use in golf carts, being impractical for portable and other alternate uses.

The U.S. Pat. No. 5,286,089 issued on February 1994 to Goldman, discloses a Seat cushion for alleviation of perineal and rectal discomfort, comprising a base and two upwardly projecting elongate foam members parallel and spaced apart, forming a channel along the center thus relieving the pressure on the perineal area, but said invention neither provides adjusting options for the angle and space between the foam upper members nor provision for the attaching of a compact warming device.

There are also patents of many other cushions claiming useful features for the perineal and prostate discomfort and there are commercially available cushions with the same purpose, as well as supporting harnesses for male perineal use with diverse purposes and embodiments, but none provides all the features and particularities of the innovative adjustable split cushion and the supporting harness intended to support in secure comfortable position the warming device of the present invention and associated therewith.

A common disadvantage inherent to the other electrically powered prior art inventions mentioned above in relation to the present one, is that most cannot be fully portable, requiring the user to remain stationed in one place, usually with adequate privacy, while portability is an important feature because to be effective, the heat application treatment to parts of the human body, usually needs prolonged time and repeated cycles, what is advantageously accomplished when the heating devices are portable and appropriate for use either at home, at the workplace, while driving and even while moving.

The U.S. Pat. No. 7,526,344 B2 issued on April 2009 to Kim, discloses a Medical device for treating prostate diseases including the use of near-Infrared L.E.D.'s, with one embodiment combined with a vibrator that is inserted into the rectum to radiate infrared rays toward the prostate providing a massage function at the same time.

That embodiment of said invention, can be used directly at home by the patient himself, likely providing an effective alleviating treatment for the prostate, but requires an uncomfortable and relatively invasive and bothersome procedure that can hurt the user if not properly handled.

However, other embodiment of said Medical Device for Treating Prostate Diseases by Using Near-Infrared L.E.D.'s is the closest prior art to the present invention, since it is placed externally at the user's perineal area, but mainly relies upon L.E.D.'s near infrared wavelengths for its heat emission, while the present invention mainly relies on ceramic based far infrared wavelengths heat emission, which are capable of deeper penetration through the body tissues.

Furthermore, the shape and size of said embodiment for perineal external use is substantially bulky and is more uncomfortable to accommodate at the user's perineal area, therefore it is not so suitable for portable use and for the user to wear it either over or under his clothes as the present invention.

There are also commercially available devices like the Negative Ion Magnetic Heating Far Infrared Ray Belt for Prostate, marketed under the brand BMK by a South Korea merchant, which claims to base its effect upon far infrared heat emission, although relies on L.E.D.'s as heating source, which also features a supporting harness for portable use.

None of those two mentioned above devices are so compact as the object of the present invention and therefore are less comfortable to accommodate to the user's perineal area, as well as too expensive and sophisticated, demanding a relatively high level of expertise and skill to operate and regulate their performance since they have complicated controls, risking a malfunction or improper setting that could harm the user and/or the device itself.

In general, in these field, there are many prior art inventions which are variations of those examples mentioned and briefly discussed above, but according to our search, so far no invention or design has been submitted claiming the specific features of the “Compact perineal warming device for personal non-invasive portable and stationary use to prevent and alleviate prostate discomfort” object of the present invention, which is very reliable and user friendly because it is very compact and conveniently built, shaped and dimensioned to easily and comfortably fit at the user's perineal area, exposed over or concealed under the clothes, for portable or stationary use, and without requiring any kind of expertise or skill because it does not need any kind of setting whatsoever.

Thereby, its novelty should be understood as being distinguished from all related prior art patents and the incorporated references are only provided for enabling support of several ways in which this kind of device can be made.

### OBJECT OF THE INVENTION

The prostate gland is an important part of the male reproductive organs located in the interior of the abdomen, in the lower part of the pelvis a few centimeters above the perineum, which in males is the short, narrow and irregular area located between the thighs from behind the base of the scrotum to the anus, being the exterior part of the male body nearest to the prostate, and for that reason is precisely in that perineal area where this innovative warming device is intended to be placed.

The object of the present invention is a personal warming device that provides advantageous features in relation to prior art inventions, particularly for the external non-invasive application of concentrated thermal relieving treatment for some of the frequent prostate gland ailments symptoms in the human male, such as Prostatitis, Benign Prostate Hyperplasia (BPH), Prostate Cancer and others.

Said purpose is fulfilled transferring concentrated heat focused to the prostate gland through the skin and adjacent tissues, even through the clothes fabric, in order to increase the gland temperature to prevent or alleviate the pain and other uncomfortable symptoms associated with the ailments mentioned.

### BRIEF SUMMARY OF THE INVENTION

The warming device according to the present invention is externally placed at the perineal area, located between the thighs immediately behind the base of the testicles scrotum, being is capable of transmitting heat to the prostate gland and surrounding tissues in order to increase the temperature in that area promoting a significant relieving effect of the painful and discomfort symptoms associated with prostate diseases common in the human males, while being safe for the skin and adjacent healthy tissues.

The main operating element of this innovative device is the warming unit, mainly integrated by a little electrical resistance embedded in a rigid ceramic prismatic case providing a protective shell, conveniently shaped and dimensioned, that allows it to be gently pressed to the perineal area of the male body, in order to achieve an effective contact for the heat transmission toward the prostate gland.

Due to the way that this device is used, the warming unit is conveniently encased within a soft elastic insulating component made of a suitable soft flexible plastic insulating material, that surrounds the ceramic electrical resistance, preventing unnecessary heat loss and at the same time protecting the adjacent areas of the thighs from any discomfort that could be caused by contact with the hard and hot surfaces or the ceramic heating electrical resistance.

This invention is very reliable and safe for the user, because its temperature and power requirements are substantially low and the electrical voltage involved is also very low, of the order of 5 volts or less, thereof there is no risk of damage or accident due to sweating or any body fluid, while, in addition, the warming unit protective insulating encasement is conveniently sealed to prevent any fluid penetration to its electric components.

This device can be comfortably held in place by means of the associated special strap-on supporting harness, which keeps the warming unit at the user's perineum without interfering with the user's normal physiological needs and activities and positions, either standing, sitting, lying in bed, walking, working out, driving, traveling, etc., thereby it can be worn while performing most normal daily actions.

In addition, since the warming unit operates in a sensitive area from the hygienic point of view, it can be covered with an outer thin flexible encasement made of any suitable material such as some disposable sanitary finger cots, etc.

This innovative device can be easily manufactured using commercially available materials and accessories, and can be easily powered, when used in portable or stationary modes either by rechargeable batteries or power banks like those used to recharge many models of electronic gadgets and mobile phones, or when used in stationary mode when the user is stationed in one place for some time, sitting or lying in bed or driving, from stationary standard power outlets also through standard power supply adapters like those available to recharge many electronic gadgets and mobile phones, as well as from USB ports available in personal computers

Its simple embodiments, instead of using sophisticated and delicate electronic circuits, the heating element and the power source are balanced so that the heat emitted is the necessary and sufficient to increase the temperature within known safe limits for the skin and other healthy tissues between 108° F. (42.2° C.) and a maximum of 113° F. (45° C.) which are known to be safely tolerated by the skin and other surrounding tissues without requiring any kind of additional setting or regulation by the user.

Nevertheless, more complex designs can be developed adding some other accessories such as an on/off switch, automatic timer switch, battery charge level indicator, power stepped selection, etc.

A timer switch can be provided, either with adjustable or fixed settings, in order to establish predetermined periods of time for the warming treatment cycles, as well as other elements may be added, such as L.E.D. or buzzer signals to alert when the batteries need to be recharged.

This device does not require any special expertise or training, it is easy to put it on and easy to be held securely in either in vertical or horizontal position, either at rest or moving, using the special strap-on supporting harness or by means of the special adjustable split cushion when sitting, both accessories being part of the invention, and practically there is not any risk of harm or damage in its handling and when wearing it, thereby assuring a very long working life, except for the batteries that eventually may require replacement or recharging.

If battery packs are used as energy source, the recommended batteries are AA size, preferably 4 in series in this case to provide approximately the required power, which are conveniently interconnected and installed inside a protective case provided with a removable cover for the eventual replacement of the batteries, and with some kind of fastening means to attach it to the belt or pants at the user's waist.

The batteries case can also be provided with an electronic circuit including an L.E.D. and/or a buzzer to alert the user when the batteries need to be recharged, and can also be provided with a simple on/off toggle switch or even with an electronic circuit to supply different power voltages in order to get different temperature levels or heating rates.

To recharge the batteries and/or to directly power the device when the user is not moving, can be used a common commercially available power adapter, preferably with AC 110 V input and output around DC 5 V and capacity for an electric current of at least 1.5 Amp.

For the interconnection of the components of the present invention, flexible fine gauge electric cables of the required lengths are used, as well as USB standard class 2A plug/receptacle combinations or DC coaxial connections, to take advantage of the abundant availability of those types of electronic connections and power sources and devices and adapters of that kind that are currently found everywhere at work places, at home, in cars, etc.

Several prototypes of the present invention according to this specification, including all its accessories have been successfully manufactured and tested, fully verifying its main object, which is to provide an effective portable therapeutic device to alleviate the painful and discomfort symptoms associated with common prostate ailments, as well as its feasibility, reliability and performance.

The mentioned objects, features and advantages of the present invention, as well as others, will become apparent to those skilled in the art, after the examination of the illustrative drawings and the detailed description of the present invention and the appended claims.

It is to be understood that this invention is not limited in its application to the details of construction and to the arrangement of the components in the following description or as illustrated in the drawings.

The invention herein described is feasible with other embodiments and of being used in practice and carried out in various ways which will be obvious to those skilled in the art. Also it is to be understood that the phraseology and terminology employed herein are for the purpose of description and should no be regarded as limiting.

As such, those skilled in the art will appreciate that the conception upon which this disclosure is based may readily be utilized as a basis for the design of other structures, methods and systems for carrying out the purpose of the present disclosed device.

It is important, therefore, that the claims be regarded as including such equivalent construction and methodology insofar as they do not depart from the spirit and scope of the present invention.

### BRIEF DESCRIPTION OF THE DRAWINGS

FIG. 1 is a general view in isometric perspective of an embodiment of the warming device, which is the main component of the “Compact perineal warming device for the “compact perineal warming device for personal non-invasive portable and stationary use to prevent and alleviate prostate discomfort”.

FIG. 2 illustrates the same embodiment of the warming device with several alternate power sources.

FIG. 3 is an electrical diagram of the same embodiment and a battery power supply for portable use.

FIG. 4A is a top view of the assembled warming unit of the embodiment of the warming device shown in FIG. 1.

FIG. 4B is a side view of the assembled warming unit of FIG. 4A

FIG. 4C is an axial longitudinal section view of the warming unit view shown in FIG. 4B.

FIG. 4D is a cross section view of the warming unit view shown in FIG. 4B.

FIG. 5 is an exploded view in isometric perspective of the components of the warming unit previously shown in the FIG. 4A thru FIG. 4D.

FIG. 6 is a lateral profile view of a user's body illustrating the position of the warming unit of the invention placed at the perineum.

FIG. 7 illustrates in an isometric perspective view an embodiment of the strap-on supporting harness of the invention to hold in place the warming unit when using it standing, walking or lying in bed but vertically shown.

FIG. 8 is an exploded view in isometric perspective illustrating the components of the strap-on supporting harness of the invention shown in FIG. 7.

FIG. 9A illustrates in an isometric perspective view an embodiment of the special split cushion of the invention attached to a seat to keep in place the warming unit when the user is sitting at home, at the working place or driving a car.

FIG. 9B is an exploded view in isometric perspective of the components of the special split cushion shown in FIG. 9A.

FIG. 10 illustrates in an isometric perspective view a base fabric pad with its upper surface providing “Velcro” loop properties, which can be used to keep in place the Warming Unit over soft seats.

### DETAILED DESCRIPTION OF THE INVENTION

The “Compact perineal warming device for personal non-invasive portable and stationary use to prevent and alleviate prostate discomfort” object of the present invention comprises a special warming device with an electrical energy source and several supplementary accessories to improve its performance and facilitate its use, being its innovative feature the way to apply its thermal effect to relieve or alleviate the discomfort that often affects the human male associated with several common ailments of the prostate gland.

The innovative and most important component integrating the “Compact perineal warming device for personal non-invasive portable and stationary use to prevent and alleviate prostate discomfort” is the warming device, which comprises as main element a warming unit, which in the preferred embodiment described is an electrical resistor embedded in ceramic, with appropriate electrical resistance and electrical power, as well as conveniently covered, shaped and dimensioned to be comfortable accommodated at the user's perineal area to safely provide a combined radiation and conduction heat transfer towards the prostate gland located at short distance above in the interior of the lower part of the bowel inside the human male body, in order to elevate the prostate temperature, thereby contributing to reduce the gland inflammation caused by several common male ailments, in a way that is less bothersome and uncomfortable than the traditional hot water heat baths and much less invasive, much less sophisticated and much easier to operate than other prior art invention with the same purpose, as previously explained.

For more efficient and secure use, the warming unit of the warming device includes a thermal insulation shield and water proofing elements covering the electrical resistor, that at the same time provide an ergonomic and soft encasement around it to make its use between the user's thighs more comfortable, particularly when the user moves or change his posture.

Other important components of the “Compact perineal warming device for personal non-invasive portable and stationary use to prevent and alleviate prostate discomfort” are: the energy source, which for portable or stationary mode of use is a common battery bank or pack, like those often used to charge some electronic gadgets, while for stationary mode of use exclusively is a common power adapter connected to any standard home or workplace power network or any other suitable power source, in all cases supplying the appropriate electric power at the proper low voltage required for the safe operation of the warming device, both from the electrical and heat exposure points of view.

When using batteries as energy source, 3 or 4 of the rechargeable type AA 1.5 V connected in series are preferred and the same alternate common power adapters used for stationary use exclusively can be also used to recharge the batteries when necessary.

The warming unit must be securely, comfortable and hygienically held in place by means of the specially designed strap-on supporting harness associated herewith as special accessory part of the present invention as will be shown further.

When the device is in portable use, the user can move freely, walking, driving and even working out and running, because the rechargeable portable battery pack or bank can be held in place at the waist level by any suitable means.

In the preferred embodiment of the invention discussed the nominal power voltage of the energy source is the standard low DC 5 V, thereof suitable power adapters are widely available, either for home and workplace stationary use or for use when driving a vehicle or travelling.

All elements and components of the present invention, as those integrating the described embodiment, as well as in all their possible variations are widespread available in the market, or are very easily manufactured, and the manufacturing and assembling involved are performed by means of very well known manufacturing processes in the field.

In the following figures, the position of the same or equivalent components or parts of components are designated with the same final two digits at the right, but consecutively increasing for each different element, while the first digit at the left corresponds to the figure in which the specific element is first depicted.

Most main features of the “Compact perineal warming device for personal non-invasive portable and stationary use to prevent and alleviate prostate discomfort” are explained in the detailed description of the preferred embodiment shown by the illustrative figures; however, as it is obvious, the embodiments of the present invention and all its components may be much more diversified than those illustrated.

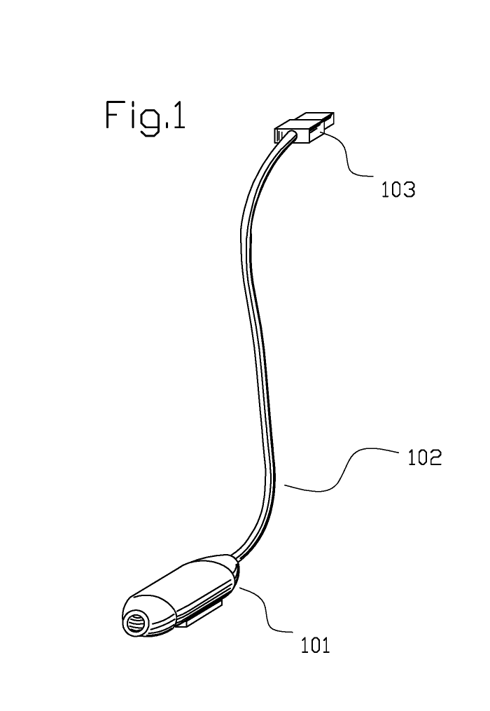

FIG. 1 depicts an isometric view of a preferred embodiment of the warming device, which is the main component of the invention, with three interconnected parts: the warming unit 101; the interconnection flexible electric cable 102 and the connecting power plug 103, to connect it to a power source at a safe low voltage.

In this embodiment, the power plug 103 is a male USB 2A plug, so the device can be powered, either directly or through a conventional USB extension cable, from any USB 2A DC 5V female receptacle available, either by a portable power bank, desk personal computer towers, laptops and car consoles, or any standard AC 110 V power network outlet using commercially available adapters, as well as any other suitable electrical energy source.

However, the connection means can be of any other standard systems combinations, such as DC low voltage coaxial plug/socket, etc.

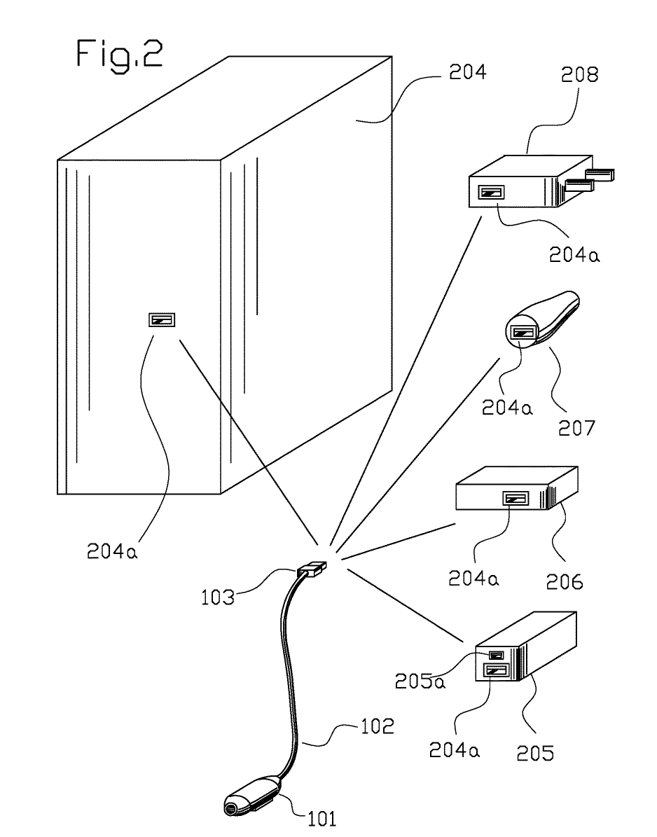

In FIG. 2 are illustrated several possible alternate power sources for the warming device shown in FIG. 1 connecting its USB male plug 103 directly to USB 2A female ports or through different types of power adapters.

For stationary use, the device power plug 103 may be directly connected to a DC 5 V USB 2A female port 204a, usually available in laptops and desktops PC towers 204.

Nevertheless, in this embodiment of the invention the preferred power source can be a portable rechargeable power bank 205 to use the device either in stationary or portable manner, concealed if desired, which in addition to the standard USB 2A female port 204a is also provided with a mini USB female port 205a to recharge it.

Alternatively, the power source can be a battery pack 206, provided with a USB 2A port 204a and comprising a commercially available portable battery case, wherein are encased the required batteries, that in this embodiment would be four AA batteries not visible in this figure, rechargeable or disposable, concealed inside the case 206.

Said battery pack case 206 comprises a removable cover, necessary for the replacement of depleted disposable batteries or old rechargeable batteries, and is also provided with an interrupting switch for operation convenience, both details being standard features of said battery cases and therefore not particularly shown in the figure.

In addition, both the power bank 205 and the battery pack 206, in order to keep them securely engaged in a convenient position when the user moves, may be provided with some sort of holding elements, such as but not limited to, small “Velcro” hook stickers.

This device can be used also while driving, directly powered from a USB port if available in the vehicle console or by means of the DC 12 V to DC 5 V USB adapter 207.

When used in stationary mode, the energy source for this device can be the power network adapter 208, like those charging adapters used to charge some mobile phones and electronic gadgets with a DC 5V USB 2A female port 204a, being the one illustrated in this embodiment for plugging in to a standard AC 110 V power network outlet either at home or at the workplace.

In addition, when necessary or convenient, a conventional extension USB cable of appropriate length can be used to interconnect both, the power source and the warming unit.

For recharging the power bank 205 or the batteries of the battery pack 206, it is necessary a standard charging USB cable, which is connected either to the car power adapter 207 to use as power source either a standard car DC 12 V power outlet or to the charging adapter 208 to use as power source a standard AC 110 V power network outlet, and if desired, the device can continue to be used in stationary mode during the charging process.

Alternatively, if necessary, any of both power adapters 207 and 208 can be also serve to recharge some mobile phones and electronic gadgets.

All connections may be provided with some sort of locking elements, such as but not limited to, a small “Velcro” strap, in order to secure them but easy to unlock to disconnect the device.

Although in the illustrated embodiment all connections depict safe low voltage DC 5 V USB connection systems, any other safe low voltage type of connections can be used, such as DC 5 V coaxial connections or any other type of safe and suitable connection.

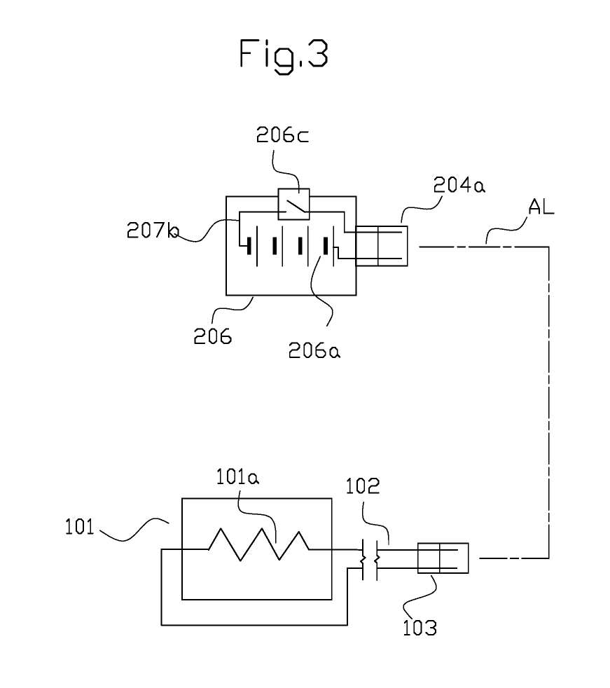

In FIG. 3 is depicted the electrical diagram of this embodiment of the invention showing schematically the essential components, although many others can be added optionally, such as an automatic timer, etc.

In said diagram is shown the warming unit 101 of FIG. 1, containing the electrical resistor 101a with the power cable 102 and a connector 103 that in this embodiment is a USB type 2A male or plug connector at the free end of the cable 102.

Also illustrated is the battery pack 206, encasing four batteries 206a connected in series, as well as the wiring 206b including a switch 206c and a USB 2A female port 204a.

Conventional electronic circuitry can be added inside the battery pack case to indicate the batteries charge level by means of an L.E.D. or other element.

In this diagram, the line AL represent the electrical connection path between the components, and although the connectors illustrated are of the USB type 2A, obviously any appropriate standard type can be used, as for example, standard DC low voltage coaxial plugs/sockets.

When the batteries are rechargeable, either an additional USB port and/or a conventional USB adapter and an appropriate charging cable, are required, using any suitable energy source combined with any of the power adapters 207 and 208 of FIG. 2, or any other charging adapter available.

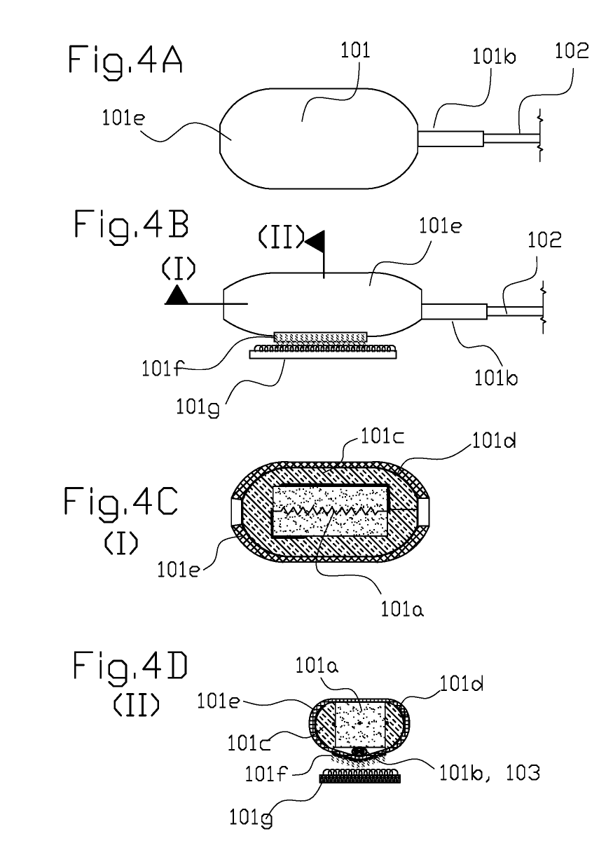

FIG. 4A is a top outer view of an assembled embodiment of the warming unit 101, which is the distinctive and main component of the invention, with the cable protecting sleeve 101b and a portion of the flexible power cable 102.

In this embodiment, the outer surface of the warming unit is covered by the insulating protecting cover 101e.

FIG. 4B is a lateral view of the same embodiment shown in FIG. 4A indicating the cutting planes for an axial longitudinal section view (I) and for a transversal section view (II), as well as the cable protecting sleeve 101b, the power cable portion 102 and the outer cover 101e.

In this view is also visible a detachable mounting element 101f used in this embodiment, consisting in a “Velcro” like touch fastening hook patch, securely adhered to the outer cover 101e at the bottom of the warming unit 101, while a matching flexible hook patch 101g, below, must be securely attached, either sticking or sewing it, onto a supporting outer base not shown in this figure, in order to hold the warming unit 101 securely at the right position under the user's perineum.

FIG. 4C shows the longitudinal section (I) previously indicated in FIG. 4B, illustrating the relative longitudinal position of some of the warming unit components for this embodiment of the warming unit of the invention: the ceramic embedded heating electrical resistor 101a; the elastic insulating lateral protection 101c; the insulating covering film 101d and the outer cover 101e.

The resistor 101a has the shape, dimensions and electrical resistance conveniently selected after multiple experiments and tests to safely achieve the main objectives of the present invention, providing the necessary heat radiation to the user's perineal area in order to prevent and alleviate the prostate discomfort associated with common male diseases in a very user friendly fashion just by plug in the device to the power source.

The flexible and elastic insulating lateral protection 101c, in this embodiment, consists in a relatively thick layer of PVC foam narrow strip surrounding the sides and opposite ends of the resistor.

The insulating covering film 101d, in this embodiment, consists in a ring of polyolefin electrical insulating material wrapped around the insulating strip 101c.

The outer cover 101e in this embodiment consists in a hypo-allergenic polyolefin shrink tube electric cable sleeve of the required size that encases all around the warming unit outer surface, providing an end opening for the cable protective sleeve 101b and the power cable portion 103 seen in FIGS. 4a and 4B.

FIG. 4D shows the transversal section (II) previously indicated in FIG. 4B, illustrating the same components seen in FIG. 4C, as well as the previously shown in the FIG. 4B touch fastening elements 101f and 101g, that in this embodiment are “Velcro” class hook and loop patches, respectively, shown attached to each other.

The main component of the warming unit is the electrical resistor 101a, located at the interior central part of the warming unit 101, which is an electrical resistance that gets hot when an appropriate electric current circulates through it, and is embedded into a rigid ceramic case in such a manner that when connected to its electrical energy source and gently pressed to the skin at the user's perineum, the warming unit 101 generates short wavelength infrared heat emission from its ceramic hot surface, known to be able of a better penetration through diverse materials and particularly through the organic tissues between the prostate gland and the perineum.

The elastic insulating strip 101c, made of a soft elastic polymeric material provides thermal insulation at the ends and both sides of the electrical resistor and achieves three purposes thereby: reduces the undesired divergent loss of heat energy; protects from the heat the skin of the user's thighs and the back of the scrotum in contact with the sides of the warming unit, and makes more comfortable the contact of the warming unit with the inner areas of the user's thighs and the back of the scrotum.

In this embodiment of the warming unit 101, important purposes of the film 101d and the outer sleeve 101e are to keep the whole unit assembled and protected against moderate humid environment and body fluids, such as sweat, as well as to hold in place the electrical resistor 101a, providing at the same time a smooth surface easy to be cleaned and kept clean for hygienic reasons.

The whole warming unit 101, must be properly sealed and protected against liquid penetration, particularly sweat or other body fluids, by any suitable means, and not only those mentioned and used in the embodiment described.

All the components of the warming unit 101 can be made in many different ways, with many different shapes, sizes and components, and can be made of diverse materials using diverse manufacturing processes.

To keep the warming unit 101 in place when in use, it has to be attached by detachable means to the special supporting harness or to the seat base as previously said and the detachable touch fastening mounting elements 101f and 101g facilitate adjusting the position and removing the device from the supporting means when necessary for cleaning or for other purposes.

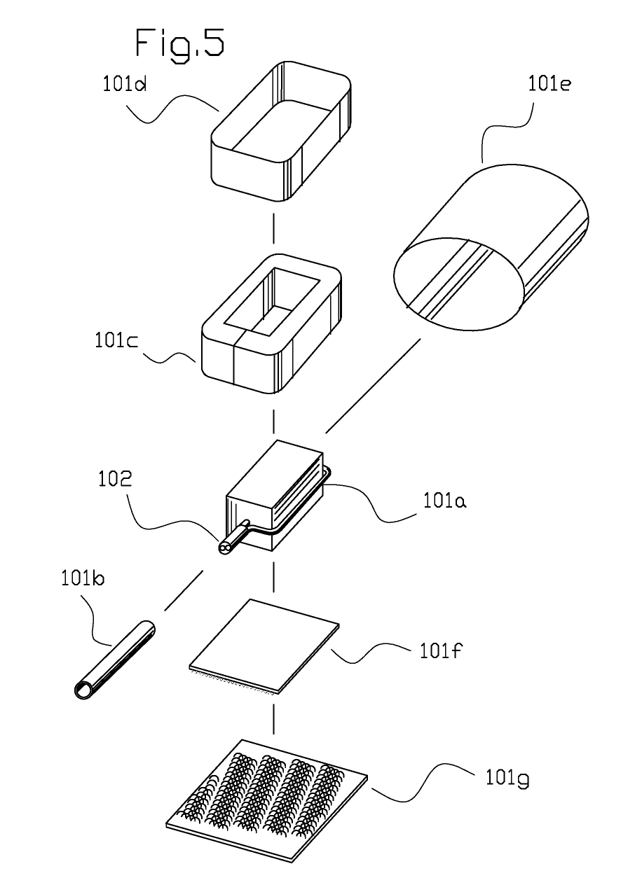

FIG. 5 is an exploded isometric view showing unassembled all those individual components 101a to 101g of the embodiment of the warming unit 101 previously shown and described in FIGS. 4A thru 4D.

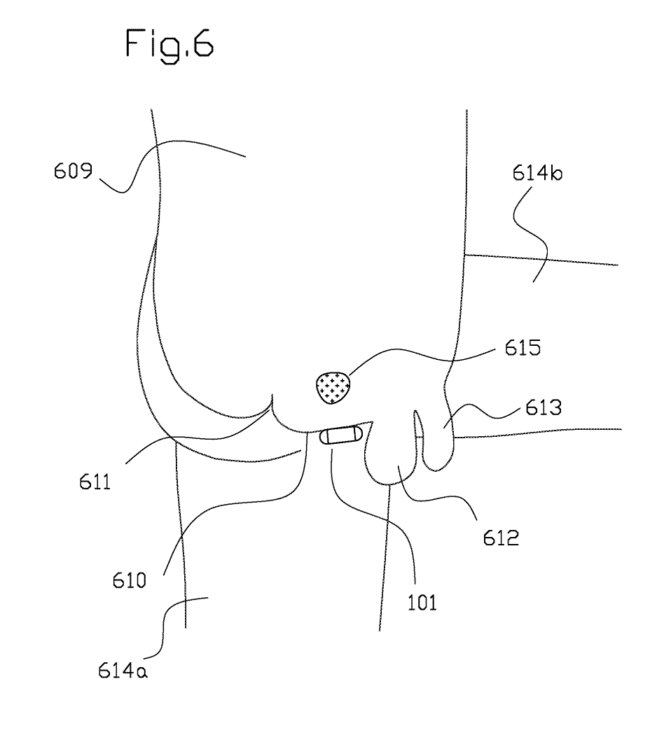

FIG. 6 illustrates in light gray lines the lateral profile contour of a male body 609, indicating the approximate position of the perineum 610, which is the area at the bottom of the pelvis, between the thighs 614a or 614b, at the sides, the anus 611 at the back and at the front, the back of the base of the testicles scrotum 612, which is an important part of the human male genitals that also include the penis 613.

In the interior of the body, at the lowest part of the pelvis and very close above the perineum 610, is located the prostate gland 615, and in this figure is illustrated the position where the warming unit 101 of the warming device must be placed in order to focus its thermal therapeutic effect to the prostate 615, helping to reduce its inflammation and alleviating the discomfort associated to several common health disorders related to this gland as already mentioned.

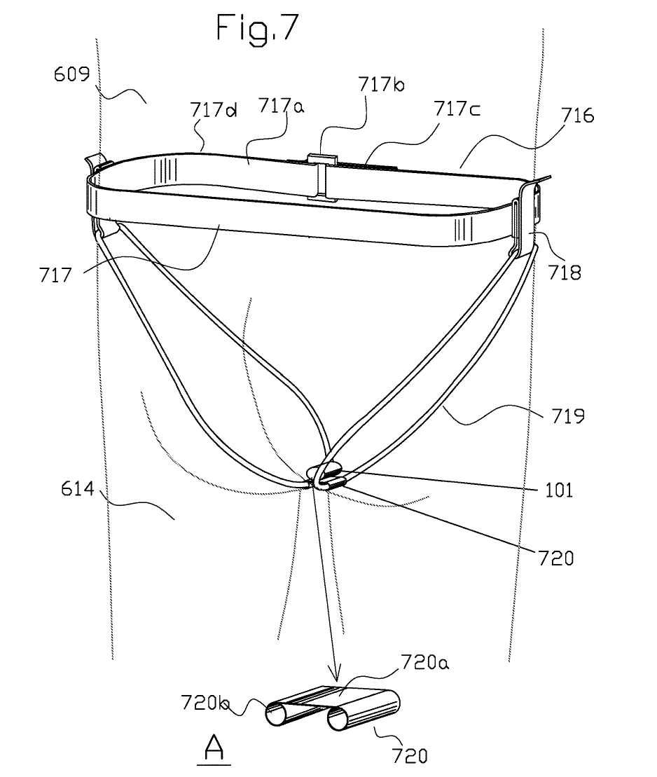

FIG. 7 is an isometric view that shows in light gray lines the contour of the human male body 609, seen from behind and slightly twisted to illustrate the adjustment of a possible embodiment of the special strap-on supporting harness 716 to hold in place the warming unit 101 of the invention, without interfering with the user's normal physiological needs, activities, movements and positions, in this case comprising an adjustable elastic waist belt 717, to which are attached two lateral adjustable holding straps 718, one at each side, each in turn holding an adjustable elastic string loop 719, for the user to wear around the upper section of each thigh 614.

The lowest segments of said loops 719 are between the thighs 614 bordering the perineum at a very little separation, and those close segments of the loops 719 are joined by a small flexible component made of an appropriate “Velcro” loop band, forming a sort of little sliding bridge 720 as shown in the magnified isometric detail view A, with a middle flat upper section 720a, with “Velcro” loop surface, that has each of its lateral edges formed like tubes 720b, wrapped around the corresponding string loop 719, in such a manner that allows to slide said bridge 720 to place it at the correct position under the user's perineum.

The warming unit 101 has a touch fastening “Velcro” hook patch 101f attached at its bottom as previously shown in FIGS. 4B, 4C and 4D, and is attached to the special strap-on supporting harness 716, fastening it onto the flat middle section 720a of the little sliding bridge 720 with “Velcro” loop surface by means of said hook patch 101f.

In this embodiment of strap-on supporting harness 716, the waist belt 717 comprises an elastic band with “Velcro” loop properties on its outer surface; a front rectangular knuckle 717b sewn to one end of the waist belt, while a “Velcro” hook patch 717c is sewn at the inner surface of the other end of said waist belt 717a in order to securely engage said belt end to the free side of the knuckle 717b at an adjustable position, according to the user's waist size, fastening it to the outer “Velcro” loop surface of said waist belt 717.

All the components are washable and reusable, and the adjusting of its components is possible by means of touch fastening elements of the “Velcro” hook/loop matching pads type for the union of all of them, but many other alternate designs, materials and construction can be adopted with a great variety of combinations and configurations for the strap-on supporting harness as a whole and for each one of its components, as long as the purpose of comfortable and reliable supporting of the warming unit 101 at the user's perineum is achieved, without interfering with the user's normal physiological needs, activities and positions.

Some of those alternate supporting components with the purpose of providing a removable adjustable mounting of the warming unit 101 to any holding attachment, can involve, for example, other touch fastening systems similar to “Velcro”, flexible magnetic patches, special fabric wrappings, and many other materials and different elements.

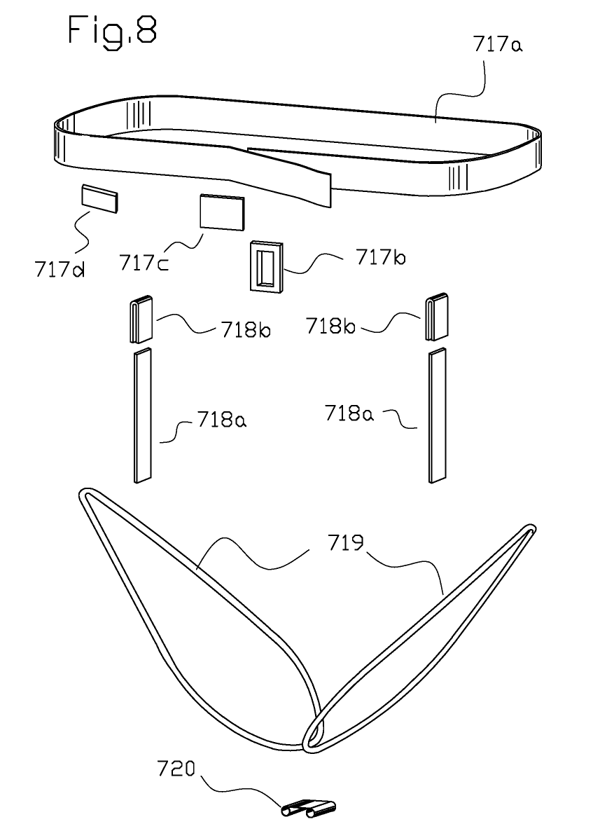

FIG. 8 is an exploded isometric view showing all the components from 717a to 720b of the embodiment of the special strap-on supporting harness 716 mentioned and described in FIG. 7, seen from the front, slightly twisted.

In this figure, in addition to the components visible in FIG. 7, are shown detached the “Velcro” hook patch 717c which is permanently sewn to the free end of the waist belt 717b in order to keep the belt adjusted to the user's waist size and the “Velcro” hook patch 717d, which is detachable and used to attach at any convenient section of the waist belt 717a, in order to hold at the desired position the power cord 102 first seen in FIG. 1 and/or the power bank 205 or the battery case 206 seen in FIG. 2.

Also disassembled are shown the “Velcro” hook patches 717b that in this embodiment are folded and sewn to the lateral adjustable holding straps 717a to keep them securely held at the sides of the waist belt 717a at the desired position at the user's hips, and at the same time securely locked when folded upward holding the elastic strings 719 shown below.

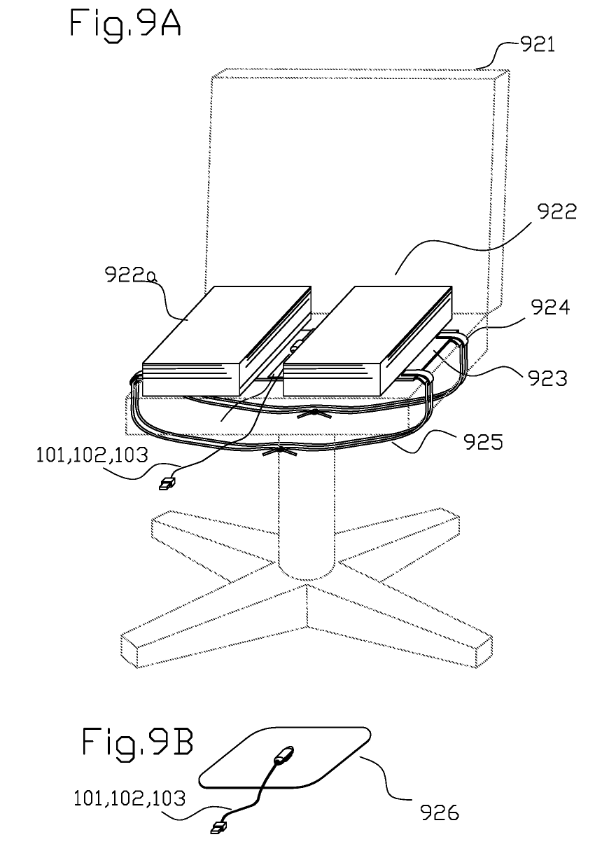

FIG. 9A is an isometric view of an assembly of a possible embodiment of the special adjustable split cushion 922 of the present invention for stationary use in sitting position, attached to a seat 921, shown in light gray lines.

This embodiment of the adjustable split cushion 922 comprises two half cushions 922a, attached in adjustable position to the seat over a base pad 923, with lateral flexible “Velcro” holding straps 924 attached to each side of said base pad 923 and bent to hold the elastic strings 925 that are wrapped around and under the seat in order to keep the split cushion 922 securely attached to the seat 921 in the desired position.

Along the middle, between both half cushions 922a, there is an empty variable space to place the warming unit 101 attached in the required position to the base pad 924 in order to keep it correctly placed under the user's perineum, while the power cord 102 and connecting plug 103 are illustrated going out from the front of the seat.

The base pad 923 upper surface has “Velcro” loop fastening properties, matching with the “Velcro” hook patch 101f of the warming unit 101 and the lateral holding straps 924, which are also made of “Velcro” hook straps facing downward to securely attach to the base pad 923, forming loops that hold the elastic strings 925 adjusted around and under the seat 921.

Each of the two half cushions 922a are attached in adjustable position to the base pad 923 by means of a “Velcro” loop strap sewn at the lower outer surface of their covers, not visible in this view.

In this embodiment, the different independent cushion components are attached to each other by means of “Velcro” like detachable means, but any other convenient detachable means can be used with the same purpose.

In the FIG. 9B is shown an isometric view of a base pad 926 that is a very simple element which, as has been found along multiple real life tests, provides the necessary and sufficient means to keep the warming unit 101, with its power cable 102 and its plug 103, in the desired place while it is used on conveniently cushioned seats, not needing the adjustable split cushion 922 shown in FIG. 9A or any other additional cushion means.

Said pad 926 consists in a substantially rectangular piece of fabric, in which its exposed upper surface provides fastening like “Velcro” loops fastening properties to attach in the desired position and conveniently secure the warming unit 101, by means of its “Velcro” hook patch 101f first shown in FIGS. 4A thru 4D.

Although said base pad 926 has been found that remains in the chosen position without any locking provision, it can be additionally secured in place by means of elements similar to the holding straps 924 and the elastic strings 925 depicted in FIG. 9.

Alternatively, the base pad can be replaced with a “Velcro” loop commercial strap with appropriate width and any convenient length, in order to hold it securely attached in the desired position on the cushion seat.

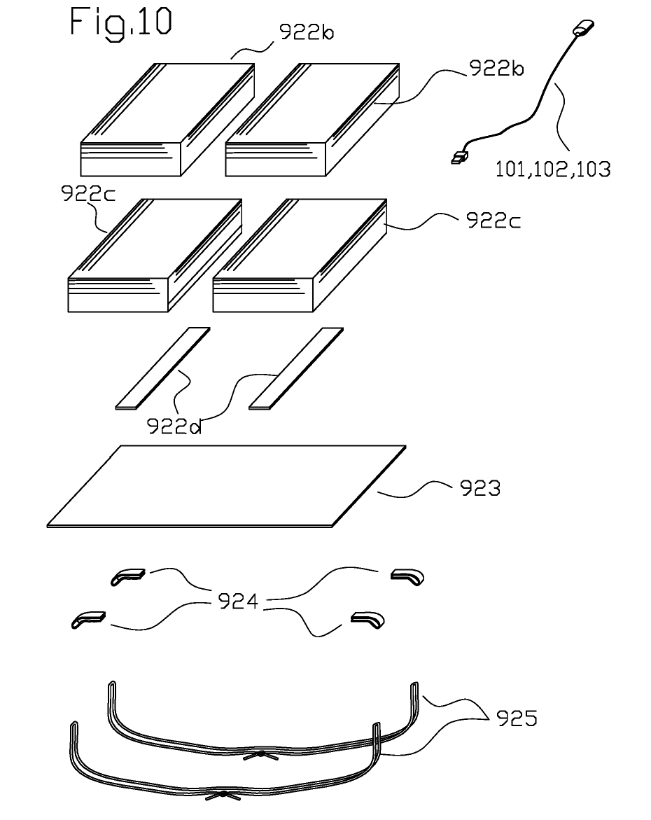

FIG. 10 is an isometric exploded view showing the components of this embodiment of the adjustable split cushion 922 shown in FIG. 9A, approximately depicting their shapes and especially showing the main parts of the two half cushions 922a previously shown in FIG. 9A: the outer covers 922b made of fabric, the inner elastic inserts 922c, in this case polyurethane elastic rectangular thick pads; the detachable means 922d, consisting in “Velcro” hook bands, which when assembled are sewn to the lower outer surface of the covers 922b; the base pad 923, consisting in a rectangular piece of fabric with its exposed upper surface providing appropriate detachable fastening means similar to “Velcro” loops, in order to attach in the desired position and conveniently secured the two half cushions 922a at each side and the warming unit 101 which is attached on the central separation space; the lateral holding straps 924 made of flexible “Velcro” hook bands; and the double elastic strings 925, that are used to hold the split cushion in place, wrapping them around the seat.

The inventors manufactured and tried several prototypes of the “Compact perineal warming device for personal non-invasive portable and stationary use to prevent and alleviate prostate discomfort” according to the described embodiments, based on the configuration shown in FIG. 1 and subsequent figures, complete with the special strap-on supporting harness and the special split adjustable cushion, having been satisfactorily tested its performance, safety and reliability as well as all its features and effects.

As a result of the experimental tests of several prototypes according to the disclosed embodiments, it was confirmed that the “Compact perineal warming device for personal non-invasive portable and stationary use to prevent and alleviate prostate discomfort” effectively achieves its purpose, preventing and alleviating prostate unpleasant symptoms caused by Prostate Benign Hyperplasia (BPH) and even by Prostate Cancer, sometimes in one only session, without any negative associated effect, thereby proving the feasibility, safety and effectiveness of the invention, and to the inventors' best knowledge it is an innovative user-friendly device to apply heat therapy to the prostate gland.

The illustrations of the embodiments described herein are intended to provide a general understanding of the structure of the various embodiments, but these illustrations are not intended to serve as a complete description of all of the elements and features of apparatus and systems that utilize the structures or methods described herein.

Additionally, the illustrations are merely representational and may not be drawn to scale. Certain proportions within the illustrations may be exaggerated, while other proportions may be minimized.

While the fundamental characteristics and features of the invention have been shown and described herein with reference to particular embodiments thereof, a plurality of modifications, various changes and substitutions are implied in the foregoing disclosure and it will be apparent that in some instances, some features of the invention may be employed without a corresponding use of other features without departing from the scope of the invention.

It should be also understood that such various substitutions, modifications and variations as well as many other embodiments may be made by those skilled in the art upon reviewing the disclosure. Therefore other embodiments may be utilized and derived from the disclosure, such that structural and logical substitutions and changes may be made without departing from the spirit and scope of the invention.

The above disclosed subject matter is to be considered illustrative, and not restrictive, and the appended claims are intended to cover all such modifications, enhancements, and other embodiments which fall within the true spirit and scope of the present disclosed subject matter. Thus, to the maximum extent allowed by law, the scope of the present disclosed subject matter is to be determined by the broadest permissible interpretation of the following claims and their equivalents, and shall not be restricted or limited by the foregoing detailed description.

The Abstract is provided to comply with 37 C.F.R. .sctn.1.72(b) and is submitted with the understanding that it will not be used to interpret or limit the scope or meaning of the claims. In addition, in the foregoing Detailed Description of the Drawings, various features may be grouped together or described in a single embodiment for the purpose of streamlining the disclosure.

This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description of the Drawings, with each claim standing on its own as defining separately claimed subject matter.

Consequently all modifications, variations and substitutions are included within the scope of the invention as defined by the claims.

What is claimed is:

1. A compact perineal warming device for personal non-invasive portable and stationary use to prevent and alleviate prostate discomfort, which is used without assistance, comprising a warming device, which in turn comprises a compact electrical warming unit and interconnection means for operatively connecting said warming unit to an electrical energy source, wherein said warming unit has smooth rounded outer shape and is substantially small, for applying heat comfortably and securely placed at the perineum of the male body, which is the outer body area nearest to the prostate gland, and said warming unit comprising a plurality of ceramic electrical resistor heating elements associated therewith, that emit concentrated infrared radiation heat at wavelengths that penetrate substantially deep through the body tissues focused to reach the prostate gland within a substantially short period of time and increasing said prostate gland temperature within safe healing limits, thereby preventing and alleviating the discomfort and other unpleasant symptoms at the prostate caused by several common male ailments.

2. The device of claim 1, wherein in order to make it therapeutically effective, as well as safe and user friendly, said electrical resistor of said warming unit is balanced with said electrical energy source to provide a maximum continuous temperature within 108° F. (42° C.) and 113° F. (45° C.) known to have healing effects and be safely tolerated by the human skin for limited periods of time and to turn it on the user only has to plug in said warming unit to said electrical energy source thereof.

3. The device of claim 1, wherein in order to improve its efficiency and comfort, said ceramic electrical resistor heating elements are surrounded by a thermal insulating shield made of a soft and elastic insulating polymer.

4. The device of claim 2, wherein said soft elastic polymer for said thermal insulating shield surrounding the electrical resistor is a substantially thick polyvinyl chloride foam insulating strip.

5. The device of claim 1, wherein in order to protect it against body fluids and liquid spills, said warming unit has an encapsulating flexible outer cover that has all openings and joints hermetically sealed, providing at the same time a hygienic smooth rounded outer surface.

6. The device of claim 4, wherein said sealing flexible cover is a hypo-allergenic waterproof polyolefin heat shrink tube.

7. The device of claim 1, wherein in order to keep said warming unit securely held in the right working position further comprises a detachable touch fastening pad securely attached at the bottom of the outer surface of said warming unit.

8. The device of claim 1, wherein in order to reduce the electrical energy consumed and to make it safe for the user, has a substantially small power of less than 1 Watt and operates at safe low voltage of not more than 5 V like existing electronic gadgets.

9. The device of claim 1, wherein said interconnection means comprise a fine gauge flexible electric cable with a connecting element at its free end, and in order to provide compatibility with the existing electrical standard accessories, said connecting element matches the USB 2A male/female combinations with low voltage of DC 5 V.

10. The device of claim 1, wherein said electrical energy source is a conventional portable rechargeable battery power bank that is provided with standard DC 5V connection means and the case of said portable rechargeable power bank is also provided with detachable holding means, in order to keep said portable power bank securely held in its working position.

11. The device of claim 1, wherein for stationary use at home and at the workplace said electrical energy source is a standard AC 110 Volts power network outlet through a standard AC 110V to DC 5V converting adapter.

12. The device of claim 1, which is also used by professional drivers that remain sitting in vehicles for long periods of time, using as electrical energy source a vehicle console DC USB 2A 5 V power port.

13. The device of claim 12, wherein, alternately, said electrical energy source is a vehicle console standard DC 12 V power outlet through a standard DC 12V to DC 5V car power adapter.

14. The device of claim 1, further using an automatic timer switch to turn off the power supply in order to limit automatically the time of operation of said warming unit.

15. The device of claim 1, wherein in order to hold said warming unit secured at the user's perineal area in portable mode use without interfering with physiological needs, further comprises a non-allergenic adjustable strap-on supporting harness.

16. The device of claim 15, wherein said adjustable strap-on supporting harness comprises an elastic waist belt adjustable to the user's waist size, with the outer surface of said elastic waist belt provided with detachable touch fastening means properties in order to securely attach to it two lateral adjustable holding straps, one at the right side and other at the left side of the user's waist, being each of said straps folded to hold an elastic string, hanging one at the right side and the other at the left side of the user's hip, and each of said elastic strings forms a loop that embraces around the corresponding thigh at its union with the trunk of the body, being the closest and lowest segments of both of said elastic strings located directly under the perineum of the user's body and joined to each other by a flexible sliding little bridge, that provides a sliding adjustable base to which is attached said warming unit by matching detachable touch fastening means.

17. The device of claim 1, wherein for stationary use sitting on substantially hard seats, further comprises a split cushion with two independent cushion sections with a plurality of shapes and dimensions separately attached over a common base pad with a plurality of shapes and dimensions by means of detachable fastening means, leaving at the middle between said independent cushion sections an adjustable space of separation, in order to prevent uncomfortable and unhealthy pressure at the user's perineal region, and at the same time providing a lower level central surface to attach by detachable fastening means said warming unit at said empty space to said base pad to securely hold said warming unit under the user's perineum.

18. The device of claim 17, wherein said two independent cushion sections have substantially rectangular shape, each being provided with washable covers, which in turn are provided with detachable touch fastening means at their bottom outer surface in order to keep said cushion sections attached to said base pad at an adjustable position in order to provide said middle space of separation between said independent cushion sections with the space and angle selected by the user and, in turn, said base pad is held to the seat by detachable touch fastening means keeping said adjustable split cushion securely attached to the seat.

19. The device of claim 17, wherein for stationary use sitting on soft seats, a base pad of a plurality of shapes and sizes is placed over the seat in order to provide a base to hold said warming unit attached by detachable fastening means at the correct position under the user's perineum, and also using detachable touch fastening means to attach said base pad at the seat.
